(19) United States
(12) Patent Application Publication
(10) Pub. No.: US 2024/0142400 A1
(43) Pub. Date: May 2, 2024

(54) BIOELECTRONIC SYSTEM FOR RARE CELL SEPARATION AND APPLICATION THEREOF

(71) Applicants: Yu-Sheng HSIAO, Taipei City (TW); PHALANX BIOTECH GROUP, INC., Zhubei City, Hsinchu County (TW)

(72) Inventors: Yu-Sheng HSIAO, Taipei City (TW); Shih-Ming TSAI, Zhubei City, Hsinchu County (TW)

(21) Appl. No.: 18/548,262

(22) PCT Filed: Mar. 2, 2022
(86) PCT No.: PCT/CN2022/078795

§ 371 Date: Aug. 29, 2023

Related U.S. Application Data
(60) Provisional application No. 63/156,083, filed on Mar. 3, 2021.

(51) Int. Cl. G01N 27/327; G01N 33/543

(52) U.S. Cl. CPC G01N 27/3275; G01N 33/5438; G01N 2333/70539; G01N 2333/726

(57) ABSTRACT

A bioelectronic system for rare cell separation and an application thereof. The bioelectronic system comprises: an electrode; a conductive polymer layer located on a surface of the electrode; a conductive polymer fiber layer located on the surface of the conductive polymer layer not in contact with the electrode; and a rare cell capturing material located the surface of the conductive polymer fiber layer not in contact with the conductive polymer layer. The conductive polymer layer has a thickness of 10-2000 nanometers. A method for rare cell separation can be provided using the bioelectronic system, and includes: introducing a biological fluid containing a rare cell into the bioelectronic system to capture the rare cell; and providing an electrical stimulus by using the electrode of the bioelectronic system to release the captured rare cell.

### PRIORITY CLAIM

The present application is a National Phase entry of PCT Application No. PCT/CN2022/078795, filed Mar. 2, 2022, which claims priority from U.S. Provisional Patent Application 63/156,83, filed Mar. 3, 2021, the disclosures of which are hereby incorporated by reference herein in their entirety.

### FIELD OF THE INVENTION

The present invention relates to a bioelectronic system for cell separation, particularly a bioelectronic system for rare cell separation, which is effective in separating rate cells and thus useful in various technical fields, including the technical fields of materials, electronics, chemistry, biopharmacology, obstetrics and gynecology, etc.

### BACKGROUND OF THE INVENTION

Cell-free pregnancy tests have many problems, including that the gene fragments adopted by the tests are insufficient to provide an overall genetic analysis result. Cell-based pregnancy tests can solve this problem. However, cell-based pregnancy tests also have a problem in that targeted cells may be lost during the tests. In general, the problem of cell-based pregnancy tests is that the targeted cells obtained by a separating step are insufficient in quantity and purity.

There are some patents and literatures attempting to solve this problem, by trying to separate targeted cells from liquid biological specimens by the following method: first, using antibodies or aptamers to recognize specific targeted proteins on the targeted cells; then, retaining the targeted cells in a specially-designed repository, such as a centrifuge tube or microfluidic channel containing magnetic beads; finally, separating the targeted cells to conduct a further genetic analysis. In the above method, the antibodies for capturing targeted cells can be EpCAM, HLA-g, bHCG, etc., and the chemical bonds of these antibodies can be surface-functionalized for capturing targeted cells on various substrates. The common bond that is generally used for modifying a substrate includes biotin and avidin/streptavidin, 1-(3-dimethylaminopropyl)-3-ethylcarbodiimide hydrochloride (EDC) and N-hydroxysuccinimide (NETS), a silane coupling agent, and the like, and the substrate that is generally used includes a silicon wafer, a glass substrate, a polyester fiber substrate, a polycarbonate substrate, a polystyrene substrate, an acrylic substrate, and the like.

In the aforementioned method, the means for the targeted cell separation step are very limited, including directly precipitating and separating the magnetic beads carrying targeted cells or cutting the selected region from the chip using a laser dissection microscope. Other means for the targeted cell separation step include separating on chips, such as by using a temperature-reactive material or breaking the bonding between the targeted cells and chip through a specific reactive agent.

However, these known methods cannot effectively release targeted cells and usually have shortcomings in high cost, low yield, etc.

### SUMMARY OF THE INVENTION

An objective of the present invention is to provide a bioelectronic system, which can effectively capture and release targeted cells to realize fast separation and cell purification. It can reduce the risks of block, lost and non-specific binding caused when a liquid biological specimen is used in the detection. The bioelectronic system comprises:
an electrode;
a conductive polymer layer, which is on a surface of the electrode and has a thickness of 10 to 2000 nanometers (nm), particularly a thickness of 50 to 1000 nm;
a conductive polymer fiber layer, which is on a surface of the conductive polymer layer that is not in contact with the electrode; and
a rare cell-capturing material, which is on a surface of the conductive polymer fiber layer that is not in contact with the conductive polymer layer.

In some embodiments of the present invention, the conductive polymer layer comprises a conductive polymer selected from the group consisting of polythiophene, poly(p-phenylene vinylene), polyacetylene, polypyrrole, polyaniline, and combinations thereof. The conductive polymer layer preferably comprises poly(3,4-ethylenedioxythiophene):poly(styrenesulfonate) (PEDOT:PSS).

In some embodiments of the present invention, the conductive polymer fiber layer comprises a conductive polymer selected from the group consisting of polythiophene, poly(p-phenylene vinylene), polyacetylene, polypyrrole, polyaniline, and combinations thereof. The conductive polymer fiber layer preferably comprises PEDOT:PSS.

In some embodiments of the present invention, the conductive polymer fiber layer has a thickness of 200 to 5000 nm.

In some embodiments of the present invention, the electrode is a transparent electrode, such as an indium tin oxide (ITO) electrode.

In some embodiments of the present invention, the rare cell-capturing material is a poly(L-lysine-graft-ethylene glycol) copolymer layer and a surface of which is modified by streptavidin and biotinylated antibody (PLL-g-PEG-biotin). The biotinylated antibody is preferably selected from the group consisting of biotinylated anti-HLA-g antibody, biotinylated anti-EpCAM antibody, biotinylated anti-bHCG antibody, and combinations thereof.

Another objective of the present invention is to provide a method for separating rare cells, comprising: introducing a biofluid containing rare cells into the bioelectronic system as described above to capture the rare cells; and providing electrical stimulation via the electrode of the bioelectronic system to release the captured rare cells.

To render the above objectives, technical features, and advantages of the present invention more apparent, the present invention will be described in detail regarding some embodiments hereinafter.

### BRIEF DESCRIPTION OF THE DRAWINGS

FIG. 1 schematically shows an embodiment of the bioelectronic system for rare cell separation according to the present invention, wherein C represents capacitance, C_{f} represents the capacitance of the conductive polymer fiber layer, and Cᵢ represents the capacitance of the conductive polymer layer;

FIG. 2 shows a flowchart of the production of an embodiment of the bioelectronic system for rare cell separation according to the present invention;

FIG. 3 shows a flowchart of the use of an embodiment of the bioelectronic system for rare cell separation according to the present invention;

FIG. 4A shows a cyclic voltammetry curve (CV curve) of electrodes with different thicknesses of conductive polymer layers, wherein the thicknesses of the conductive polymer layers are 78 nm, 87 nm, and 123 nm, respectively;

FIG. 4B shows charge capacity densities (CCDs) of electrodes with different thicknesses of conductive polymer layers, wherein the thicknesses of the conductive polymer layers are 78 nm, 87 nm, and 123 nm, respectively;

FIG. 4C shows an impedance plot of electrodes with different thicknesses of conductive polymer layers according to electrochemical impedance spectroscopy (EIS), wherein the thicknesses of the conductive polymer layers are 78 nm, 87 nm, and 123 nm, respectively;

FIGS. 5A and 5B, respectively show a CV curve and an EIS impedance plot of electrodes with different coatings, wherein ITO represents an ITO electrode without coating, PL represents an ITO electrode with a conductive polymer layer of PEDOT:PSS, NF represents an ITO electrode with a conductive polymer fiber layer of PEDOT:PSS, and PL-NF represents an ITO electrode with a conductive polymer layer of PEDOT:PSS and a conductive polymer fiber layer of PEDOT:PSS on the conductive polymer layer;

FIG. 6A shows the current response of the bioelectronic system of the present invention using cyclic voltammetry in 80 cycles;

FIGS. 6B to 6F show the CV curves of the bioelectronic system at 1^{st}, 20^{th}, 40^{th}, 60^{th}, and 80^{th} cycles, respectively;

FIG. 7A schematically shows the ITO electrodes with a conductive polymer layer of PEDOT:PSS (PL) coated by PLL-g-PEG (P) repeatedly, wherein PL′, PL″ and PL′″ respectively represent the electrodes that were subjected to electrical stimulation once, twice, and thrice, and (PL/P), (PL′/P) and (PL″/P) respectively represent the electrodes coated by PLL-g-PEG;

FIG. 7B shows the zeta potential of the aforementioned electrodes at different cycles of electrical stimulation;

FIG. 7C shows the zeta potential of electrodes with different coatings, wherein PL represents an ITO electrode with a conductive polymer layer of PEDOT:PSS, PL-NF represents an ITO electrode with a conductive polymer layer of PEDOT:PSS and a conductive polymer fiber layer of PEDOT:PSS on the conductive polymer layer, (PL-NF)/P represents a PL-NF electrode coated by PLL-g-PEG, and (PL-NF)′ represents a PL-NF electrode where (PL-NF)/P was subjected to electrical stimulation once and followed by detaching of PLL-g-PEG;

FIGS. 8A to 8E schematically show separating JEG-3 cells from a mixture of Hela cells and JEG-3 cells by using the bioelectronic system for rare cell separation according to the present invention;

FIG. 9 shows a fluorescence image of an electrode of the bioelectronic system according to the present invention, wherein (a) shows the PLL-g-PEG-biotin coating on the electrode, (b) shows JEG-3 cells captured on the electrode, and (c) and (d) shows the electrode before or after conducting electrical stimulation, respectively;

FIG. 10 shows a fluorescence image of cells obtained by the separation of the bioelectronic system of the present invention, wherein (a) shows a cell image in a bright field, (b) shows a cell image under fluorescence microscopy, and (c) and (d) show images of JEG-3 cells (blue fluorescence) and Hela cells (green fluorescence) obtained from the partially enlarged white square in FIG. 10(b).

### DESCRIPTION OF REFERENCE SIGNS IN THE DRAWINGS

C: total capacitance
C_{f}: capacitance of conductive polymer fiber layer
Cᵢ: capacitance of conductive polymer layer.

### DESCRIPTION OF THE PREFERRED EMBODIMENT

The following paragraphs will further describe some of the embodiments of the present invention regarding the drawings; however, without departing from the spirit of the present invention, the present invention may be embodied in various embodiments and should not be limited to the specific embodiments described in the specification.

In the drawings, the size of each element or region may be exaggerated and not drawn to scale for illustrative purposes.

Unless it is additionally explained, the expression “a,” “an,” “the,” or the like recited in the specification and claims should include both the singular and plural forms.

The effect of the present invention over the prior art is to provide a bioelectronic system with an improved structure, and the bioelectronic system can quickly and effectively capture targeted cells in a biological specimen and release targeted cells, thereby achieving effective separation of targeted cells. The targeted cells particularly refer to rare cells in a biological specimen. The details of the bioelectronic system of the present invention and the application of the bioelectronic system in rare cell separation are as follows.

### 1. Bioelectronic System for Rare Cell Separation

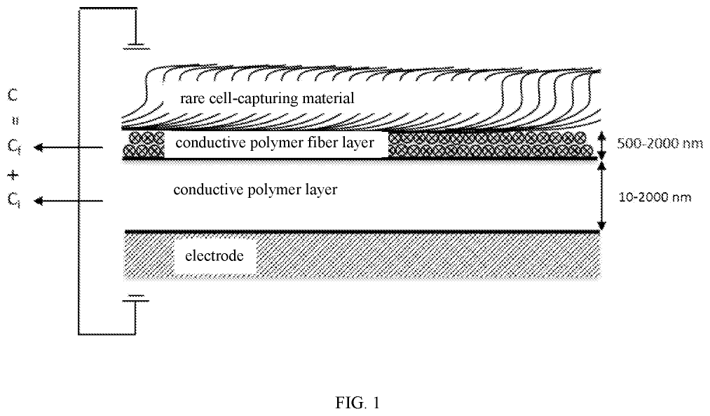

FIG. 1 schematically shows an embodiment of the bioelectronic system for rare cell separation according to the present invention. As shown in FIG. 1, the bioelectronic system for rare cell separation, according to the present invention, comprises: an electrode; a conductive polymer layer, which is on a surface of the electrode and has a thickness of 10 to 2000 nm; a conductive polymer fiber layer, which is on a surface of the conductive polymer layer that is not in contact with the electrode; and a rare cell-capturing material, which is on a surface of the conductive polymer fiber layer that is not in contact with the conductive polymer layer.

The bioelectronic system of the present invention can provide an excellent capture rate of targeted cells and release rate of targeted cells by including the conductive polymer layer and conductive polymer fiber layer. Without being limited by theory, it is believed that this is because of the synergic effect of using the conductive polymer layer to provide a capacitance particularly suitable for cell release and using the conductive polymer fiber layer to provide high contact surface area.

To provide a suitable capacitance, the thickness of the conductive polymer layer is 10 to 2000 nm, preferably 50 to 1000 nm, such as 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, or 990 nm, or within a range between any two of the values described herein. If the thickness of the conductive polymer layer is lower than the above ranges, the bioelectronic system cannot provide a reasonable release rate of targeted cells. On the other hand, if the thickness of the conductive polymer layer is higher than the above ranges, the transparency of the bioelectronic system will be reduced, thereby increasing the difficulty in observation through microscopy. In a preferable embodiment of the present invention, the conductive polymer layer has a thickness of 50 to 500 nm and, more specifically, a thickness of 60 to 200 nm.

The preferable thickness of conductive polymer fiber layer is 200 to 5000 nm, such as 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, 1500 nm, 1550 nm, 1600 nm, 1650 nm, 1700 nm, 1750 nm, 1800 nm, 1850 nm, 1900 nm, 1950 nm, 2000 nm, 2050 nm, 2100 nm, 2150 nm, 2200 nm, 2250 nm, 2300 nm, 2350 nm, 2400 nm, 2450 nm, 2500 nm, 2550 nm, 2600 nm, 2650 nm, 2700 nm, 2750 nm, 2800 nm, 2850 nm, 2900 nm, 2950 nm, 3000 nm, 3050 nm, 3100 nm, 3150 nm, 3200 nm, 3250 nm, 3300 nm, 3350 nm, 3400 nm, 3450 nm, 3500 nm, 3550 nm, 3600 nm, 3650 nm, 3700 nm, 3750 nm, 3800 nm, 3850 nm, 3900 nm, 3950 nm, 4000 nm, 4050 nm, 4100 nm, 4150 nm, 4200 nm, 4250 nm, 4300 nm, 4350 nm, 4400 nm, 4450 nm, 4500 nm, 4550 nm, 4600 nm, 4650 nm, 4700 nm, 4750 nm, 4800 nm, 4850 nm, 4900 nm, or 4950 nm, or within a range between any two of the values described herein. In some embodiments of the present invention, the conductive polymer fiber layer has a thickness of 200 to 2000 nm.

In the bioelectronic system of the present invention, the conductive polymer layer or the conductive polymer fiber layer can independently have a single-layer structure or a multiple-layer structure as long as the overall thickness of the conductive polymer layer or the conductive polymer fiber layer is within the indicated ranges.

In the bioelectronic system of the present invention, the type of conductive polymer contained in the conductive polymer layer, or the conductive polymer fiber layer is not particularly limited and can be selected depending on the need. The conductive polymer contained in the conductive polymer layer can be identical to or different from that contained in the conductive polymer fiber layer. The conductive polymer layer and the conductive polymer fiber layer can independently consist of a single material or multiple materials, and the entire material of the conductive polymer layer or the conductive polymer fiber layer can be a conductive polymer, or a portion of the material of the conductive polymer layer or the conductive polymer fiber layer can be a conductive polymer. Examples of the conductive polymer include but are not limited to polythiophene, poly(p-phenylene vinylene), polyacetylene, polypyrrole, polyaniline, and combinations thereof. In an embodiment of the present invention, the conductive polymer layer, and the conductive polymer fiber layer each independently comprises PEDOT:PSS, substantially consists of PEDOT:PSS, or consists of PEDOT:PSS.

In the bioelectronic system of the present invention, the electrode type is not particularly limited and can be selected depending on the need. The preferable electrode is a transparent electrode in terms of convenience of observation. In an embodiment of the present invention, the electrode is an ITO electrode.

In the bioelectronic system of the present invention, the type of the rare cell-capturing material is not particularly limited and can be selected depending on the type of cells to be captured. Such selection can be done by persons having ordinary skill in the art based on the disclosure of the present specification and their general knowledge, and therefore will not be described in detail. In an embodiment of the present invention, the rare cell-capturing material is a poly(L-lysine-graft-ethylene glycol) copolymer layer and the surface of which is modified by streptavidin and biotinylated antibody (PLL-g-PEG-biotin copolymer layer).

In the bioelectronic system of the present invention, the above biotinylated antibody is selected depending on the type of cells to be captured. Examples of the biotinylated antibody include but are not limited to: biotinylated anti-HLA-g antibody, biotinylated anti-EpCAM antibody, biotinylated anti-bHCG antibody, and combinations thereof. For example, the biotinylated anti-HLA-g antibody can be used to capture trophoblast cells in the placenta, and the biotinylated anti-EpCAM antibody can be used to capture circulating tumor cell (CTC).

### 2. Preparation of Bioelectronic System for Rare Cell Separation

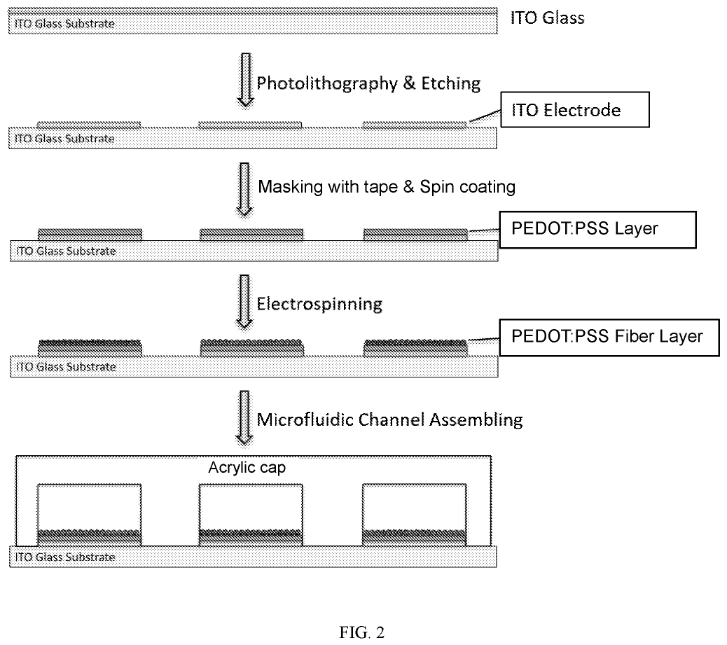

FIG. 2 shows a flowchart of the production of an embodiment of the bioelectronic system for rare cell separation according to the present invention. As shown in FIG. 2, an ITO electrode is prepared by providing a glass substrate having an indium tin oxide layer and then conducting photolithography and etching to the glass substrate. Afterwards, the electrode surface is masked by a tape and a PEDOT:PSS layer (i.e., conductive polymer layer) is formed by spin coating. Then, a PEDOT:PSS fiber layer (i.e., conductive polymer fiber layer) is formed on the surface of the PEDOT:PSS layer by electrospinning. Finally, the surface of the PEDOT:PSS fiber layer is covered by an acrylic cap to form a microfluidic channel for the flow of specimens.

To impart the bioeletronic system of the present invention the capability of capturing targeted cells, a layer of rare cell-capturing material is formed on the surface of the conductive polymer fiber layer. Generally, a conductive polymer fiber layer with a negative charge can combine with a rare cell-capturing material with a positive charge, and a conductive polymer fiber layer with a positive charge can combine with a rare cell-capturing material with a negative charge. After completing the preparation procedures as shown in FIG. 2, the rare cell-capturing material can bind to the surface of conductive polymer fiber layer by having the rare cell-capturing material flow through the microfluidic channel.

### 3. Use of Bioelectronic System for Rare Cell Separation

After forming the rare cell-capturing material on the surface of the conductive polymer fiber layer, the bioelectronic system of the present invention has the capability of capturing targeted cells (e.g., rare cells). Therefore, the present invention also provides a method for separating rare cells, comprising: introducing a biofluid containing rare cells into the bioelectronic system of the present invention to capture rare cells; and providing electrical stimulation via the electrode of the bioelectronic system to release the captured rare cells.

In particular, FIG. 3 shows a flowchart of the use of an embodiment of the bioelectronic system for rare cell separation according to the present invention. As shown in FIG. 3, after the conductive polymer fiber layer is modified by a rare cell-capturing material depending on the type of cells to be captured to form the rare cell-capturing material on its surface, the biological specimens to be analyzed can flow through the bioelectronic system of the present invention, and the targeted cells will be captured by the rare cell-capturing material. Afterwards, the electrode is energized to provide electrical stimulation to neutralize the charges carried by the conductive polymer fiber layer and rare cell-capturing material, thereby allowing the rare cell-capturing material that is combined with targeted cells detaches the surface of conductive polymer fiber layer to accomplish the release of targeted cells.

For example, in a JEG-3 rare cell separation, the rare cell-capturing material can be a poly(L-lysine-graft-ethylene glycol) copolymer layer, and a surface of which can be modified by streptavidin and biotinylated antibody (PLL-g-PEG-biotin copolymer layer).

### 4. Examples

### 4.1. Experimental material and method

1. Hela cells: human cervical cancer cells purchased from Bioresource Collection and Research Center (BCRC) of Food Industry Research and Development Institute.
2. JEG-3 cells: human placental choriocarcinoma cells purchased from BCRC.
3. Stain for Hela cells: Syto-16 stain, purchased from Thermo-Fisher Company.
4. Stain for JEG-3 cells: Hoechst stain, purchased from Thermo-Fisher Company.
5. Phosphate buffered saline (PBS): purchased from Thermo-Fisher Company.
6. ITO electrode: purchased from UNI-ONWARD Co., Ltd.
7. PEDOT:PSS: purchased from Germany Heraeus Company, product no.: PH1000.
8. PLL-g-PEG-biotin: purchased from Swiss SuSoS Company.
9. Biotinylated anti-HLA-g antibody: purchased from Invitrogen Company.
10. Streptavidin: purchased from Thermo Company, product no.: SA488.
11. Conductive polymer layer: provided by spin coating a solution containing 94 wt % of PEDOT:PSS, 5 wt % of DMSO, and 1 wt % of GOPS on an ITO electrode.
12. Conductive polymer fiber layer: provided by electrospinning with a solution containing 87 wt % of PEDOT:PSS, 10 wt % of poly(ethylene glycol) (PEO; purchased from Sigma-Aldrich Company), and 3 wt % of (3-glycidyloxypropyl) trimethoxysilane (GOPS; purchased from Sigma-Aldrich Company).
13. Cyclic voltammetry (CV): obtained by scanning from −0.8V to +0.8 V at a scan rate of 100 mV s⁻¹.
14. Electrochemical impedance spectroscopy (EIS): the frequency range is 10⁻¹ to 10³ hertz (Hz).

### 4.2. Example 1: Effect of Thickness of Conductive Polymer Layer on Electrochemical Property

The conductive polymer layers of PEDOT:PSS (hereinafter referred to as “PEDOT:PSS layer”) having different thicknesses were formed by spin coating on ITO electrodes, and the electrochemical properties of the ITO electrodes having PEDOT:PSS layers with different thicknesses were observed to evaluate the effect of the thickness of the conductive polymer layer for the bioelectronic system of the present invention.

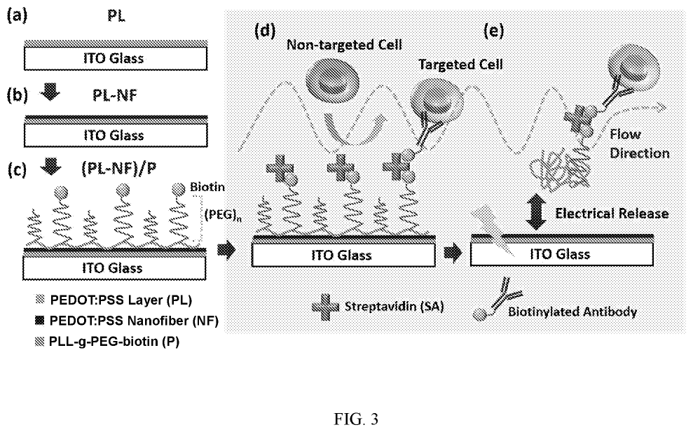

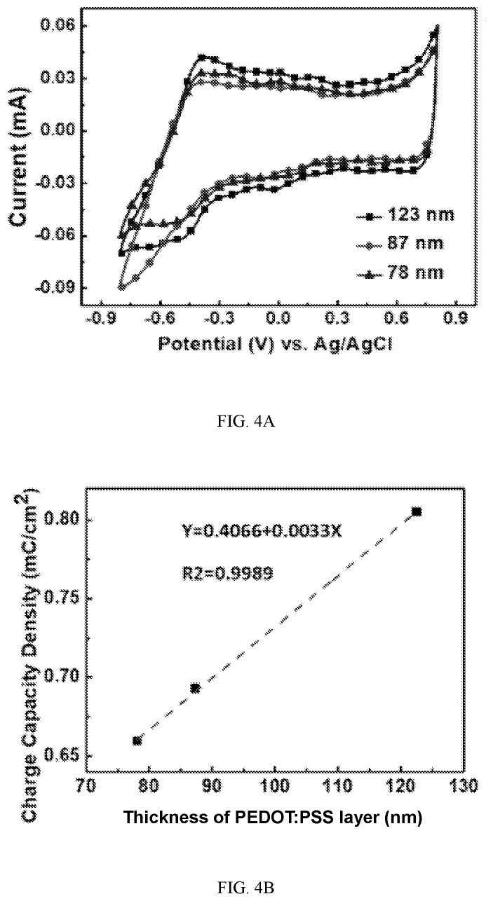

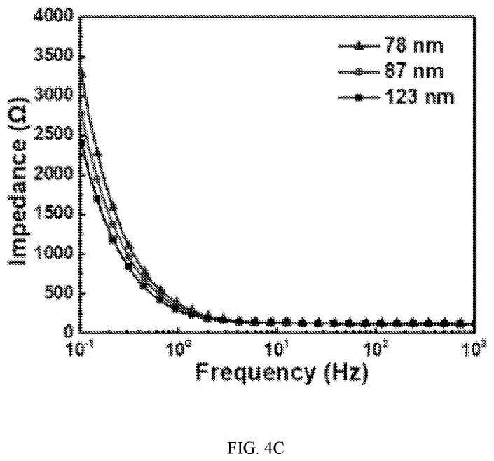

First, the charge capacity densities (CCDs) of the ITO electrodes having the PEDOT:PSS layers with different thicknesses were analyzed by cyclic voltammetry. As shown in Table 1 and FIGS. 4A and 4B, the experimental results show that the charge capacity density of the electrode increases with the increasing PEDOT:PSS layer thickness.

TABLE 1

| Thickness of PEDOT:PSS | Charge capacity density |
|---|---|
| layer (nm) | (mC/cm²) |
| 78 | 0.66 |
| 87 | 0.69 |
| 123 | 0.81 |

In addition, the ITO electrodes having PEDOT:PSS layers with different thicknesses were subjected to electrochemical impedance spectroscopy. As shown in FIG. 4C, the impedance of the electrode decreases with increasing PEDOT:PSS layer thickness.

The above experimental results show that the PEDOT:PSS layer with a certain thickness can effectively impart charge-discharge property to a thin film capacitor to control the positive-negative switch of surface charge.

### 4.3. Example 2: Effect of Coating Component on Electrochemical Property

Cyclic voltammetry and electrochemical impedance spectroscopy were used to compare the effect of using different coatings on an ITO electrode in terms of electrochemical properties. As shown in FIGS. 5A and 5B, in comparison with ITO (an uncoated ITO electrode), PL (an ITO electrode having an 87 nm thick PEDPT:PSS conductive polymer layer), and NF (an ITO electrode having a PEDPT:PSS conductive polymer fiber layer with a diameter of 247±49 nm), PL-NF (an ITO electrode having an 87 nm thick PEDPT:PSS conductive polymer layer as well as a PEDPT:PSS conductive polymer fiber layer with a diameter of 247±49 nm) has the greatest CV area and the lowest impedance, and the coating was formed by depositing a PEDOT:PSS conductive polymer fiber layer on an ITO electrode by electrospinning for 10 minutes.

The above experimental results surprisingly show that as compared to the embodiment of using a PEDOT:PSS conductive polymer layer or a PEDOT:PSS conductive polymer fiber layer alone, the embodiment of the present invention that uses a PEDOT:PSS conductive polymer layer in combination with a PEDOT:PSS conductive polymer fiber layer (i.e., PL-NF) has a synergic effect in providing electrode capacitance, and thus can significantly increase the CV area and reduce impedance. As shown in the following test results of capturing and releasing rare cells, the bioelectronic system of the present invention thereby has an excellent release rate of targeted cells.

### 4.4. Example 3: Stability Test

4.4.1. Cyclic Voltammetry Test

The long-term stability of the above PL-NF embodiment (the ITO electrode having an 87 nm thick of PEDPT:PSS conductive polymer layer and a PEDPT:PSS conductive polymer fiber layer with a diameter of 247±49 nm) was observed by multiple cyclic voltammetry scanning.

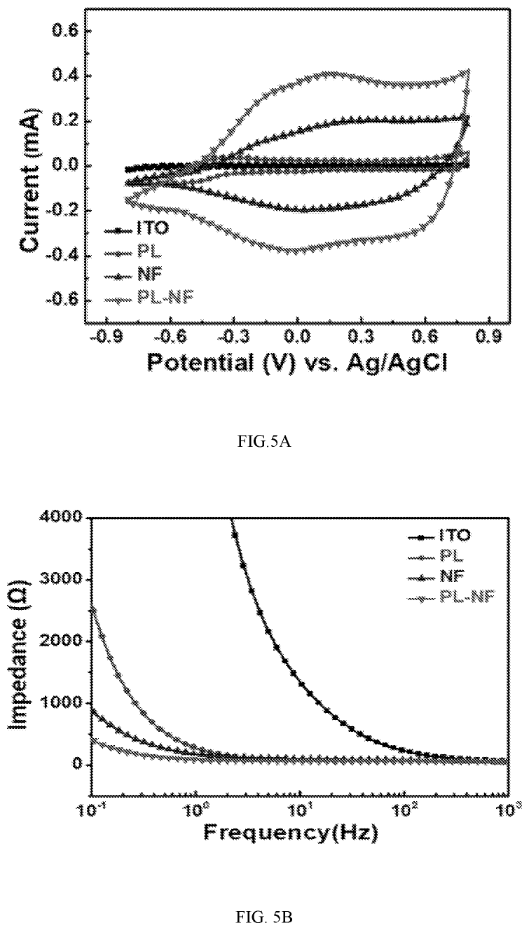

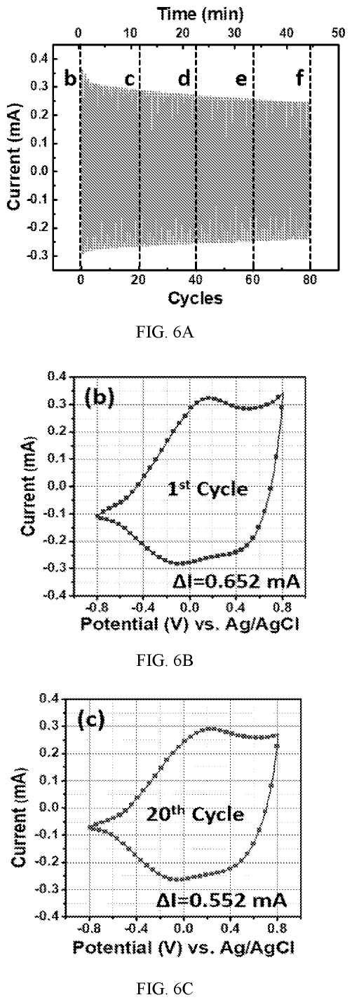

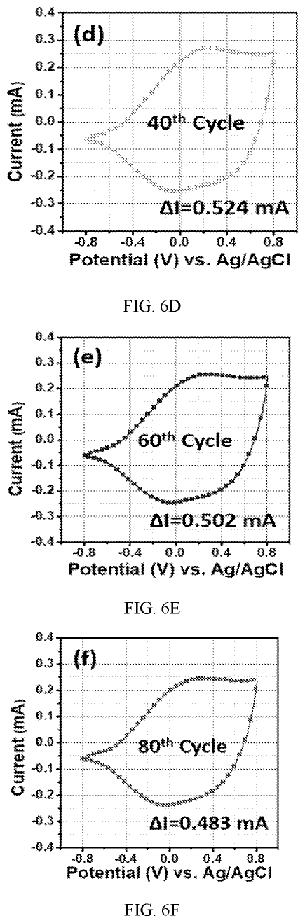

As shown in FIGS. 6A to 6F, even after 80 cycles, the current change and CV curve of PL-NF did not significantly change, meaning that the bioelectronic system of the present invention can have a great stability for long-term use.

4.4.2. Zeta Potential Test

The bioelectronic system of the present invention was used to capture targeted cells by the rare cell-capturing material on the conductive polymer fiber layer. This test is to observe zeta potential to determine the stability of the bioelectronic system of the present invention after repeated use.

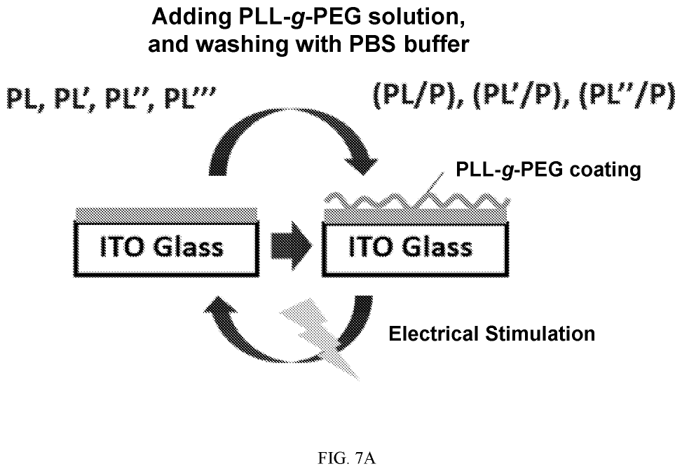

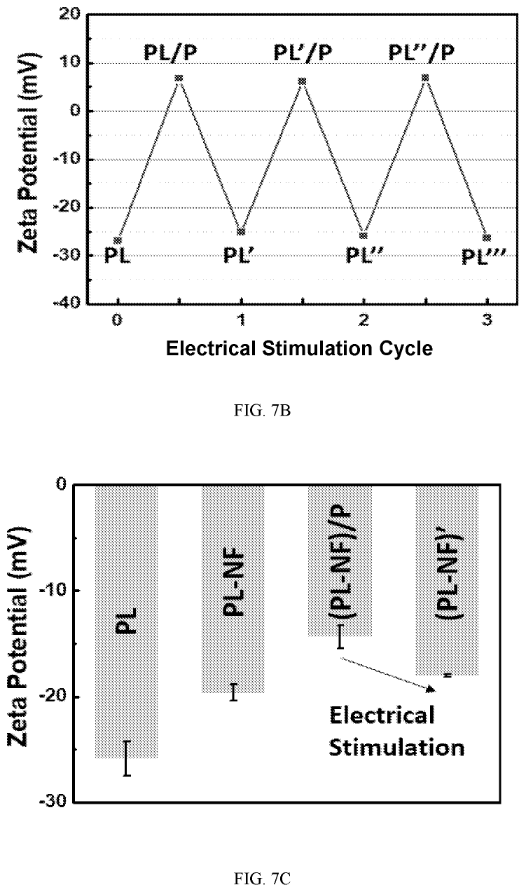

As shown in FIG. 7A, PLL-g-PEG solution was added to the electrode having the PEDOT:PSS conductive polymer (PL; the thickness of conductive polymer is 87 nm), and the electrode was washed with a PBS buffer to obtain an electrode having a PEDOT:PSS conductive polymer surface that is coated with PLL-g-PEG (PL/P). Thereafter, the coating of PLL-g-PEG on the electrode surface was detached by electrical stimulation to retrieve an electrode having a PEDOT:PSS conductive polymer surface that is not coated with PLL-g-PEG (PL′). The above steps were repeated to provide an electrode that is two times coated with PLL-g-PEG and an electrode that is three times coated with PLL-g-PEG (PL′/P and PL″/P) and the corresponding electrodes that are electrically stimulated twice and thrice (PL″ and PL′″). As shown in FIG. 7B, the electrodes having PEDOT:PSS conductive polymer after conducting electrical stimulation and recoating of PLL-g-PEG all have similar zeta potential.

The inventors of the present invention further tested the electrode having PEDOT:PSS conductive polymer and PEDOT:PSS conductive polymer fiber (PL-NF) and the bioelectronic system of the present invention ((PL-NF)/P). As shown in FIG. 7C, the bioelectronic system of the present invention after electrical stimulation ((PL-NF)′) can have zeta potential that is similar to that of the bioelectronic system before coating PLL-g-PEG (PL-NF).

### 4.5. Example 4: Capturing and Releasing Test of Rare Cells

As described above, the bioelectronic system of the present invention can be used to separate rare cells. The following will test the effect of the bioelectronic system of the present invention on capturing and releasing cells by conducting a separation of JEG-3 cells from a mixture of Hela cells and JEG-3 cells.

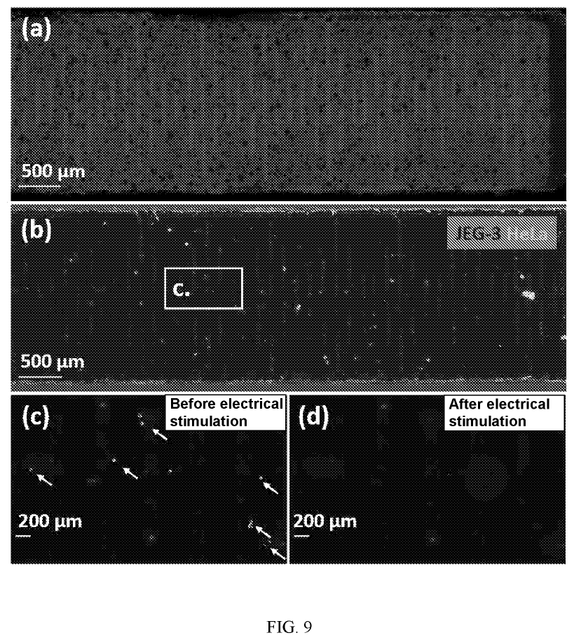

A bioelectronic system having microfluidic channels with three working electrodes (E1, E2 and E3) and one reference electrode (E4) was used, wherein the E1, E2 and E3 electrodes were ITO electrodes having the following surface coatings: a PEDOT:PSS conductive polymer layer (87 nm thick) on the surface of the ITO electrode, a PEDOT:PSS conductive polymer fiber layer (247±49 nm in diameter) on the surface of the PEDOT:PSS conductive polymer layer, and a poly(L-lysine-graft-ethylene glycol) copolymer layer on the surface of the PEDOT:PSS conductive polymer fiber layer, and a surface of the poly(L-lysine-graft-ethylene glycol) copolymer layer is modified by streptavidin and biotinylated antibody (PLL-g-PEG-biotin copolymer layer). Before conducting the test, AlexaFluor555-coupling streptavidin (SA-555) was added, and the biotinylated PLL-g-PEG coatings of electrodes in the bioelectronic system were observed under fluorescence microscopy, as shown in FIG. 9(a).

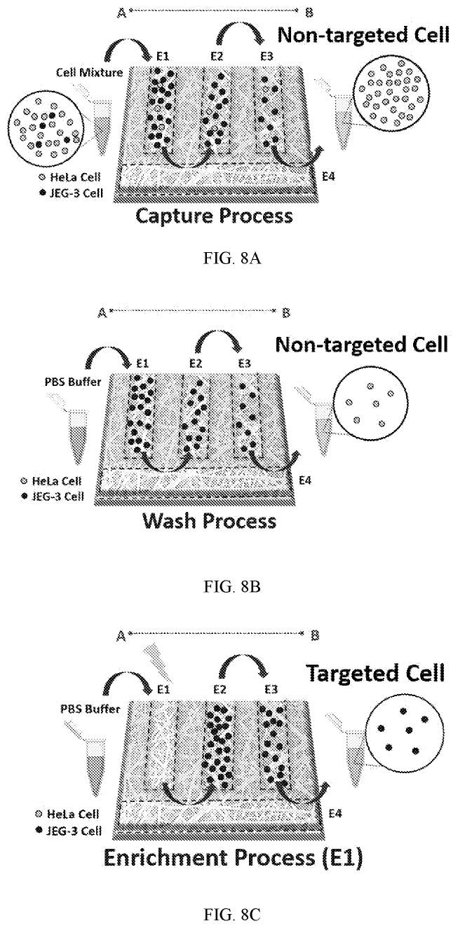

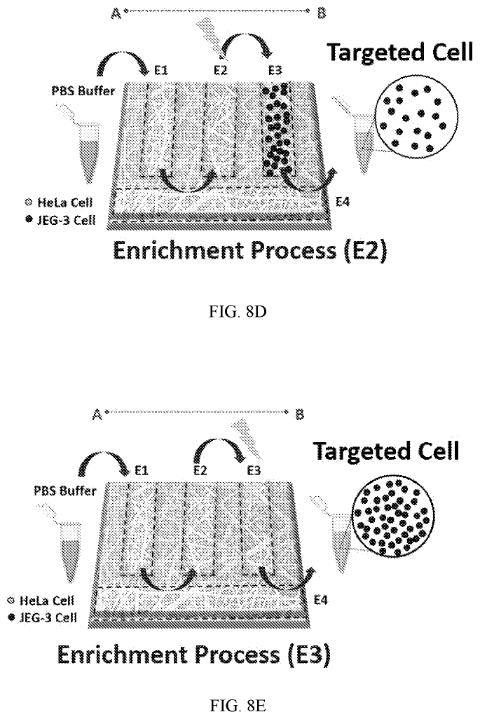

Afterwards, as shown in FIGS. 8A to 8E, a cell mixture containing 10⁶ Hela cells (non-targeted cells) and 2,000 JEG-3 cells per mL of the mixture was added into the microfluidic channels of the bioelectronic system, and the mixture sequentially flowed through E1, E2 and E3 to capture the targeted cells (i.e., JEG-3 cells). Then, the microfluidic channels of the bioelectronic system were washed with a PBS buffer, thereby further excluding the remaining non-targeted cells. Finally, E1, E2 and E3 were sequentially subjected to electrical stimulation to enrich the targeted cells.

As shown in FIG. 9(a), the electrodes in the bioelectronic system of the present invention can effectively capture targeted cells and enrich the targeted cells (JEG-3 cells, blue fluorescence) from the non-targeted cells, and the non-specific capture rate is only 3.43±0.57% (Hela cells, blue fluorescence). The white square in FIG. 9(b) was further enlarged to observe, and after electrically stimulating the electrodes, the cells indicated by white arrows in FIG. 9(c) disappeared; that is, the targeted cells were released by electrical stimulation (FIGS. 9(c) and (d)).

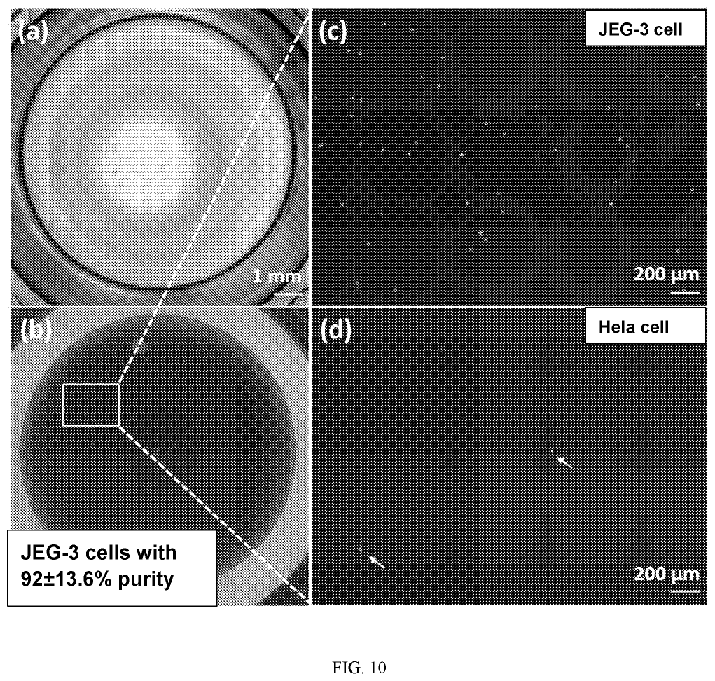

FIGS. 10(a) and (b) show images of cells collected after the separation of the bioelectronic system of the present invention under a bright field and fluorescence microscopy. The white square in FIG. 10(b) was partially enlarged to observe further, and the collected cells are mainly targeted cells (i.e., JEG-3 cells) with blue fluorescence, and only a few of the non-targeted cells with green fluorescence, wherein the purity of targeted cells (i.e., JEG-3 cells) is 92±13.6%, and the release rate is up to 65%.

The above experimental results illustrate that the bioelectronic system for rare cell separation, disclosed by the present invention, can effectively capture, and release rare cells in a specimen.

The above example is only used to exemplarily illustrate the principle and efficacy of the present invention and show the inventive features thereof rather than limit the protective scope of the present invention. Without departing from the principle and spirit of the present invention, all of the modifications and replacements which may be accomplished by any person skilled in the art fail in the scope as claimed by the present invention. Therefore, the scope of protection of the present invention is as defined in the claims as appended.

1. A bioelectronic system for rare cell separation, comprising:
an electrode;
a conductive polymer layer, which is on a surface of the electrode and has a thickness of 10 to 2000 nm;
a conductive polymer fiber layer, which is on a surface of the conductive polymer layer that is not in contact with the electrode; and
a rare cell-capturing material, which is on a surface of the conductive polymer fiber layer that is not in contact with the conductive polymer layer.

2. The bioelectronic system of claim 1, wherein the conductive polymer layer has a thickness of 50 to 1000 nm.

3. The bioelectronic system of claim 1, wherein the conductive polymer layer comprises a conductive polymer selected from the group consisting of polythiophene, poly(p-phenylene vinylene), polyacetylene, polypyrrole, polyaniline, and combinations thereof.

4. The bioelectronic system of claim 3, wherein the conductive polymer layer comprises poly(3,4-ethylenedioxythiophene):poly(styrenesulfonate).

5. The bioelectronic system of claim 1, wherein the conductive polymer fiber layer comprises a conductive polymer selected from the group consisting of polythiophene, poly(p-phenylene vinylene), polyacetylene, polypyrrole, polyaniline, and combinations thereof.

6. The bioelectronic system of claim 5, wherein the conductive polymer fiber layer comprises poly(3,4-ethylenedioxythiophene):poly(styrenesulfonate).

7. The bioelectronic system of claim 1, wherein the conductive polymer fiber layer has a thickness of 200 to 5000 nm.

8. The bioelectronic system of claim 1, wherein the electrode is a transparent electrode.

9. The bioelectronic system of claim 8, wherein the electrode is a tin oxide electrode.

10. The bioelectronic system of claim 1, wherein the rare cell-capturing material is a poly(L-lysine-graft-ethylene glycol) copolymer layer and a surface of which is modified by streptavidin and biotinylated antibody.

11. The bioelectronic system of claim 10, wherein the biotinylated antibody is selected from the group consisting of biotinylated anti-HLA-g antibody, biotinylated anti-EpCAM antibody, biotinylated anti-bHCG antibody, and combinations thereof.

12. A method for separating rare cells, comprising:
introducing a biofluid containing rare cells into the bioelectronic system of claim 1 to capture the rare cells; and
providing electrical stimulation via the electrode of the bioelectronic system to release the captured rare cells.
